(19) United States
(12) Patent Application Publication
(10) Pub. No.: US 2024/0158823 A1
(43) Pub. Date: May 16, 2024

(54) GENETICALLY MODIFIED AZOTOBACTER VINELANDII STRAINS AND METHODS OF USING SAME

(71) Applicant: REGENTS OF THE UNIVERSITY OF MINNESOTA, Minneapolis, MN (US)

(72) Inventors: Brett Barney, Minneapolis, MN (US); Alexander James Steiner, Saint Paul, MN (US)

(21) Appl. No.: 18/506,263

(22) Filed: Nov. 10, 2023

Related U.S. Application Data
(60) Provisional application No. 63/425,707, filed on Nov. 16, 2022.

(51) Int. Cl. C12P 13/02; C12N 9/00; C12N 9/88; C12N 9/90; C12N 15/74

(52) U.S. Cl. CPC C12P 13/02; C12N 9/88; C12N 9/90; C12N 9/93; C12N 15/74; C12Y 402/01011; C12Y 501/01003; C12Y 603/02; C12Y 603/02017; C12N 2800/101; C12N 2800/90

(57) ABSTRACT

A genetically modified diazotrophic microbe is genetically modified to produce γ-polyglutamic acid (γ-PGA). In one or more embodiments, the diazotrophic microbe is Azotobacter vinelandii. In one or more embodiments, the diazotrophic microbe is genetically modified to use a non-sugar carbon source.

### CROSS-REFERENCE TO RELATED APPLICATION

This application claims the benefit of U.S. Provisional Patent Application No. 63/425,707 filed Nov. 16, 2022, which is incorporated herein by reference in its entirety.

### GOVERNMENT FUNDING

This invention was made with government support under 2020-67019-31148 awarded by the National Institute of Food and Agriculture. The government has certain rights in the invention.

### SEQUENCE LISTING

This application contains a Sequence Listing electronically submitted via Patent Center to the United States Patent and Trademark Office as an .xml file entitled “0110-000706US01.xml” having a size of 5 kilobytes and created on Nov. 9, 2023. The information contained in the Sequence Listing is incorporated by reference herein.

### SUMMARY

This disclosure describes, in one aspect, a genetically modified diazotrophic microbe. In one or more embodiments, the diazotrophic microbe is Azotobacter vinelandii. In one or more embodiments, the diazotrophic microbe is genetically modified to produce γ-polyglutamic acid (γ-PGA). In one or more embodiments, the diazotrophic microbe is genetically modified to use non-sugar carbon sources.

In one or more embodiments, the microbe includes one or more exogenous genes. In one or more of these embodiments, at least one exogenous gene is a gene naturally found in Bacillus spp. In one or more of these embodiments, at least one exogenous gene encodes one or more exogenous gene products in a biosynthetic pathway that produces γ-polyglutamic acid (γ-PGA).

In one or more embodiments, the diazotrophic microbe includes a gene homologous to a member of the pgsBCAE operon from B. subtilis. In one or more of these embodiments, the diazotrophic microbe includes the pgsBCAE operon from B. subtilis.

In one or more embodiments, the diazotrophic microbe includes a deletion or disruption of one or more genes involved in the synthesis of a biopolymer. In one or more of these embodiments, the biopolymer comprises alginate or polyhydroxybutyrate.

In one or more embodiments, the diazotrophic microbe produces a unique high-molecular weight band on an SDS-PAGE gel as compared to a non-genetically modified diazotrophic microbe.

In one or more embodiments, the diazotrophic microbe is A. vinelandii.

In another aspect, this disclosure describes a method of increasing growth in an organism. Generally, the method includes co-culturing the organism with any embodiment of a genetically modified diazotrophic microbe.

In another aspect, this disclosure describes a diazotrophic microbe genetically modified to exhibit diazotrophic growth when provided with a non-sugar carbon source.

In one or more embodiments, the carbon source is a compound of the tricarboxylic acid (TCA) cycle. In one or more of these embodiments, the carbon source is malate, fumarate, succinate, 2-oxaloacetate, or citrate.

In one or more embodiments, the diazotrophic microbe has a mutation in a gene homologous to nifLA in A. vinelandii.

In one or more embodiments, the microbe has a genetic mutation to increase expression of a gene homologous to the fixABCX cluster from A. vinelandii.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Schematics of relevant molecules and genes. (A) Structures of glutamic acid, α-polyglutamic acid, and γ-polyglutamic acid (γ-PGA). (B) Structures of polyhydroxybutyrate, polyglutamate, and alginate. (C) Depiction of pgs and cap genes in Bacillus spp. The pgs and cap operons encode the polyglutamate synthase proteins, while pgdS and capD encode peptidases.

FIG. 2. Genotypes of relevant A. vinelandii strains used in this study. A. vinelandii DJ has the phb operon and alg operons intact. AZBB131 has the phb operon deleted. AZBB652 has the alg operon deleted. AZBB667 and AZBB697 are based on AZBB652 and have thepgs operon and a kanamycin cassette inserted in place of phbB. AZBB690 is based on AZBB652 and has lacZ and a kanamycin resistance cassette inserted in place of phbB. AZBB699 is based on AZBB697 and has racE from B. subtilis behind the S-layer protein promoter.

FIG. 3. SDS-PAGE gels of microbial lysates. (A) An SDS-PAGE gel of A. vinelandii and Bacillus species supernatants stained with methylene blue. Lane 1: AZBB131, Lane 2: AZBB652, Lane 3: AZBB667, Lane 4: protein ladder, Lane 5: B. natto in LB medium, Lane 6: B. natto in M9 medium, Lane 7: B. subtilis 168 in LB medium, Lane 8: B. subtilis 168 in M9 medium. (B) An SDS-PAGE gel of periplasmic extracts of A. vinelandii strains stained with methylene blue. Lane 1: protein ladder, Lane 2: AZBB131, Lane 3: AZBB652, Lane 4: AZBB667. (C) An SDS-PAGE gel of A. vinelandii supernatants stained with methylene blue. Lane 1: A. vinelandii DJ (wild-type), Lane 2: AZBB131, Lane 3: AZBB652, Lane 4: protein ladder, Lane 5: empty, Lane 6: AZBB667, Lane 7: AZBB690.

FIG. 4. Data characterizing γ-PGA production in A. vinelandii. (A) Size exclusion chromatogram of A. vinelandii supernatants. (B) Size exclusion chromatogram of AZBB667 compared to a γ-PGA standard curve. (C) Visual comparison of lyophilized supernatants of A. vinelandii strains. 50 mL volumes of supernatant were lyophilized following seven days of culture growth.

FIG. 5. Characterization of the impact of ultrafiltration on γ-PGA production in A. vinelandii strains. (A) SDS-PAGE gel of ultrafiltered AZBB652 and AZBB667 concentrates stained with methylene blue. Lanes 1 and 3: AZBB652. Lanes 2 and 5: AZBB667. Lane 3: protein ladder. (B) Size exclusion chromatogram of ultrafiltered AZBB652 and AZBB667 concentrates and as lyophilized supernatants that were not filtrated.

FIG. 6. H¹-NMR spectra of AZBB667 and γ-PGA standard. The peak at 4.8 ppm corresponds to D₂O used as a solvent. The AZBB667 spectrum is black and the γ-PGA spectrum is grey.

FIG. 7. Characterization of the impact of urea and/or glutamate on production of γ-PGA in A. vinelandii strains. (A) SDS-PAGE gel of A. vinelandii strains stained with methylene blue. Lanes 1-4: AZBB667. Lanes 6-9: AZBB667. Lane 5: protein ladder. (B) Size exclusion chromatogram of AZBB667 (Top) and AZBB697 (Bottom) under varying growth conditions.

FIG. 8. Quantification of γ-PGA production using SDS-PAGE gel and densitometry analysis. (A) SDS-PAGE gel of γ-PGA standards and A. vinelandii γ-PGA producing strains stained with methylene blue. Lanes 1-4: γ-PGA standards. Lanes 6-9: γ-PGA producing strains. Lane 5: protein ladder (B) Standard curve for densitometry analysis with sample results fitted to the equation (y=305.78x, R²=0.9856).

FIG. 9. Components used to generate transposon mutants and characterization of transposon mutants. (A) Plasmid map of pBB298 for tetracycline transposon mutagenesis. The tetracycline cassette containing Tet^{R}, R6K origin of replication, and the inverted repeats can be randomly inserted into the A. vinelandii genome at a TA site using the TA sites at the end of the inverted repeats. (B) Methylene blue stained SDS-PAGE gel of lysed AZBB667 colonies produced by transposon mutagenesis. Lane 1: protein ladder. Lane 2: AZBB667 control. Lanes 3-10: AZBB667 transposon mutants. (C) Size exclusion chromatogram. of AZBB667 transposon mutagenesis mutants. (D) Schematic of tetracycline transposon insertion into ahpC and oriented toward nitrogen regulation genes ntrB and ntrC. IR: Inverted Repeat.

FIG. 10. Characterization of the impact of racE expression on γ-PGA production. (A) SDS-PAGE gel of AZBB667 transposon mutagenesis stained with methylene blue. Lane 1: protein ladder. Lane 2: AZBB667 control. Lanes 3-10: AZBB667 transposon mutants. (B) Size exclusion chromatogram of AZBB667 transposon mutagenesis mutants.

FIG. 11. Schematics of genes relevant to several mutant A. vinelandii strains described herein. (A) Simplified schematic of the rnf1 cluster next to nifLA and the fix cluster. (B) Genotypes of relevant A. vinelandii strains that have been deregulated for ammonium production.

FIG. 12. Quantification of growth and nitrogen fixation of mutant A. vinelandii strains using different carbon sources. (A) Ammonium production (left) and optical density measurements (right) of AZBB568 grown in B Mo/Fe/S with 2 g/L of carbon source. (N≥6, Mean±SD, ammonium concentrations statistically significant at 48 hours, 72 hours, and 96 hours with p<0.00001 by one-way ANOVA for all). (B) Optical density measurements of A. vinelandii DJ (left) and AZBB568 (right) grown with 2 g/L of carbon source. (DJ: 2-oxoglutarate: N=1, Others: N=3. AZBB568: N=6-8. Mean±SD).

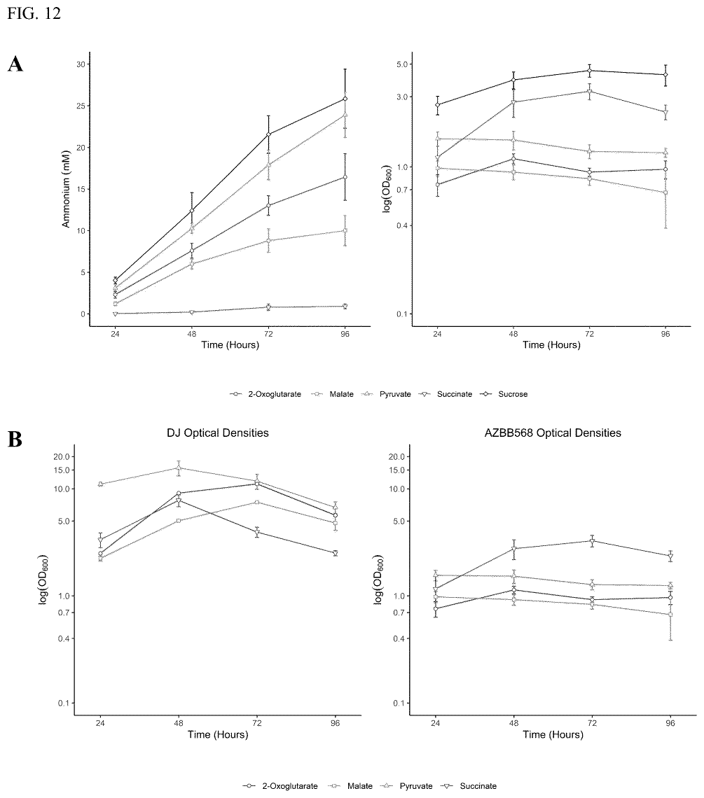

FIG. 13. Quantification of growth and nitrogen fixation of mutant A. vinelandii strains using different carbon sources. (A) Ammonium production (left) and optical density measurements (right) of AZBB696 grown in B medium supplemented with Mo/Fe/S and 2 g/L of carbon source. (N≥3, Mean±SD). (B) Ammonium production (left) and optical density measurements (right) of AZBB689 grown in B medium supplemented with Mo/Fe/S and 2 g/L of carbon source. (N≥4, Mean±SD).

FIG. 14. Ammonium accumulation in AZBB568 with various sugars. (A) Ammonium levels and cell density (optical density at 600 nm) of AZBB568 when grown on the sugars sucrose, glucose and fructose. The sugars xylose, ribose and arabinose did not result in substantial growth of AZBB568 or wild-type A. vinelandii DJ. Cultures were grown for four days at 28° C. and 180 rpm. Results shown are the average and standard deviation for day 4 (n=4). (B) Ammonium levels of AZBB568 grown in sucrose, pyruvate, α-ketoglutarate, malate, or succinate (left). Cell density (optical density at 600 nm) of AZBB568 and wild type A. vinelandii DJ when grown on sucrose, pyruvate, α-ketoglutarate, malate, and succinate (right). The organic acids citrate, oxaloacetate and fumarate did not result in substantial growth of AZBB568 or wild-type A. vinelandii DJ.

FIG. 15. A summary of TCA cycle-derived carbon sources that support extracellular ammonium excretion in AZBB568.

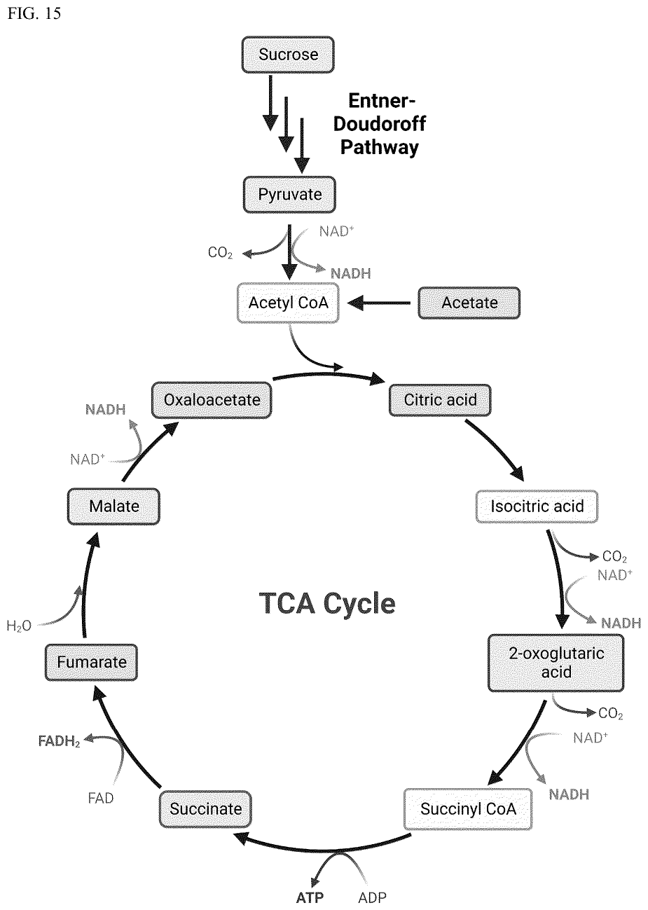

FIG. 16. Regulation of NifA expression. Under low N uridylylated GlnK cannot bind to NifL, but transcription of nif genes is only activated under high C when 2-OG is saturated. Under high N deuridylylated GlnK binds to NifL and induces formation of a GlnK-NifL-NifA ternary complex that represses transcription of nif genes regardless of C status and levels of 2-OG.

FIG. 17. Methylene blue plate of selected strains from Example 6.

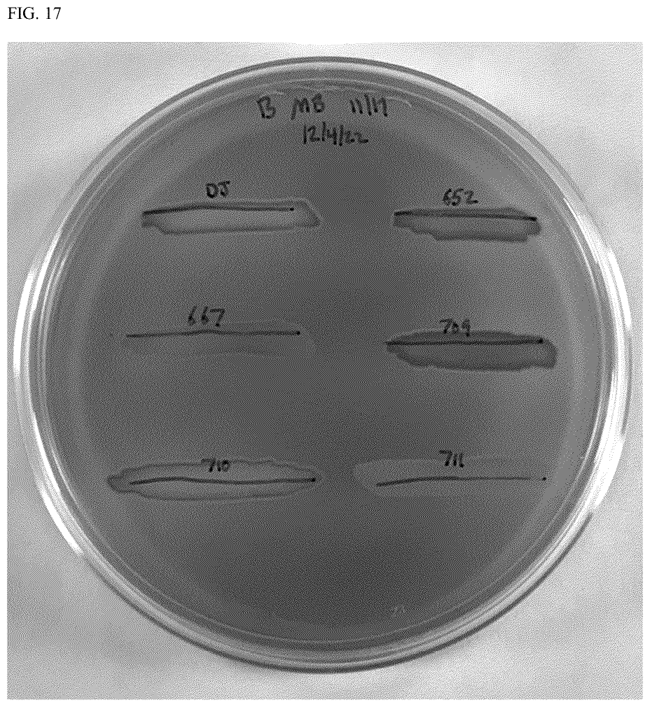

### DETAILED DESCRIPTION OF ILLUSTRATIVE EMBODIMENTS

This disclosure describes several tools involving genetically modified diazotrophic microbes for use as fertilizers. In one aspect, the genetically modified diazotrophic microbe produces γ-PGA. In another aspect, the genetically modified diazotrophic microbe can utilize carbon sources other than sugar (e.g., non-sugar carbon sources) during diazotrophic growth.

Fertilizer inputs from industrial processes such as the Haber-Bosch process come at the expense of fossil fuels. Nutrient requirements are directly linked to biomass production, and any potential increased improvement in the scale of biomass yield will necessitate a proportional increase in the demand for essential nutrients. For all photosynthetic systems—e.g., photoautotrophs such as land plants, algae, and cyanobacteria—with requisite light energy and water, nitrogen is a limiting and expensive nutrient input for aquaculture and agricultural production alike. Current nitrogen fertilizer production involves burning of fossil fuels to generate ammonia from molecular nitrogen (N₂ gas) through the Haber-Bosch process, and is extremely energy intensive. In developed countries, industrial nitrogen production is accompanied by a huge overall economic and energetic cost. In developing countries, lack of nitrogen often limits agricultural productivity, where energy and infrastructure costs impede the use of the Haber-Bosch process to produce ammonia on a large scale from atmospheric nitrogen.

Many organisms, such as bacteria, naturally fix atmospheric nitrogen into forms that can be used as fertilizer. As used herein, “nitrogen fixation” and “nitrogen assimilation” refer to the process of incorporating nitrogen from an unusable source, such as atmospheric nitrogen, into a form that can be used as a fertilizer. Organisms, such as bacteria, that fix nitrogen and their byproducts are referred to as “biofertilizers.” Many species of nitrogen-fixing soil bacteria thrive in the rhizosphere, the zone around the plant root. Beneficial bacteria in the rhizosphere are collectively known as plant-growth promoting rhizobacteria (PGPR). Examples of PGPR include bacteria from the genus Arthrobacter, Azospirillum, Azotobacter, Bacillus, Burkholderia, Enterobacter, Klebsiella, Pseudomonas, Rhizobium, Serratia, and Streptomyces.

The development of improved biofertilizers represents a unique opportunity to lower the potential economic costs and environmental impacts of current fossil-fuel-dependent industrial methods for producing ammonia-derived fertilizers. The approach described herein can circumvent the energy cost and the associated greenhouse gas emissions tied to producing and distributing nitrogen fertilizers by using a diazotrophic bacterium as a biofertilizer to provide a renewable source of nitrogen to meet the growth requirements of the associated photosynthetic species. This disclosure describes compositions and methods involving genetically-modified diazotrophic microbes that fix nitrogen and, therefore, support the growth of other organisms in co-culture. In one or more embodiments, the organism in co-culture is photosynthetic. The organism in co-culture may be a plant.

Diazotrophic bacteria may produce different nitrogen-containing compounds that can be used as fertilizer. Ammonia is a commonly produced form of fixed nitrogen that can be used by other organisms such as plants. Alternate nitrogen-containing compounds may be beneficial in different circumstances.

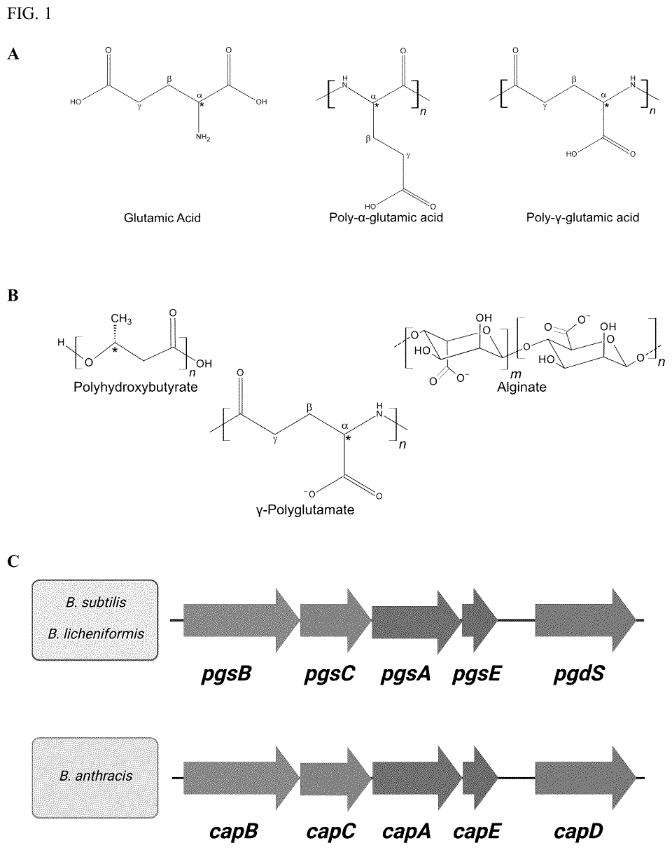

Poly-glutamic acid (PGA) is a poly-amino acid in which glutamic acid monomers are coupled to each other via amide bonds between the alpha or gamma carboxylic acid groups (FIG. 1A). γ-PGA can be microbially synthesized. Bacillus spp., such as B. licheniformis, B. subtilis, B. amyloliquefaciens, B. anthracis, and B. megaterium are known to produce γ-PGA. γ-PGA is synthesized in a ribosome-independent manner by an ATP-dependent amide ligase called γ-PGA synthase. L-glutamic acid is polymerized intracellularly through γ-amide bonds to create γ-PGA. The γ-linkages thus make this polymer resistant to α-proteases that cleave α-amino bonds. The source of L-glutamic acid for γ-PGA biosynthesis can either be exogenous or endogenous. In B. subtilis and B. licheniformis, γ-PGA synthase is encoded by four genes encoded in the pgsBCAE operon. A schematic of γ-PGA synthase genes from B. subtilis, B. lichenformis, and B. anthracis is provided in FIG. 1C.

γ-PGA may have utility as a nitrogen-containing fertilizer, as it may exhibit delayed release of nutrients relative to traditional nitrogen-containing compounds such as ammonia, meaning that nutrients are available in the soil for a longer period of time. γ-PGA may also work synergistically with other nitrogen-containing compounds. γ-PGA bound to urea fertilizer has been shown to enhance the growth of tomato plants by reducing the loss of applied nitrogen and increasing the total nitrogen content in the soil. It was also demonstrated that urea mixed with purified γ-PGA increased wheat yields more than 7% over urea fertilizer due to an increase in immobilized nitrogen content in the soil, which led to a gradual release during the late growth phase of wheat. However, most common strains of Bacillus sp., such as B. subtilis subspecies subtilis 168 and subspecies Natto BEST195 do not contain nitrogenase enzymes. Resultantly, nitrogen that is accumulated in the γ-PGA is the product of nitrogen provided to the strains in the culture medium. Since Azotobacter vinelandii can grow on a variety of carbon substrates and fixes nitrogen from the atmosphere, it is able to produce polyglutamate using only atmospheric nitrogen, while the polyglutamate then acts as a nitrogen sink and storage material.

In one aspect, the present disclosure describes a diazotrophic microbe genetically modified to produce γ-PGA. In one or more embodiments, the diazotrophic microbe may be derived from Azotobacter vinelandii. In one or more embodiments, the present disclosure describes methods to engineer A. vinelandii to produce and optionally secrete γ-PGA through heterologous expression of γ-PGA-producing genes from Bacillus subtilis.

While described herein in the context of an exemplary embodiment in which the diazotrophic microbe is A. vinelandii, the genetically modified microbes and methods described herein can involve the use of other nitrogen-fixing species. For example, as some of the diazotrophic microbes described herein are genetically modified to include genes that they do not naturally have, many other diazotrophic microbes may be compatible with inclusion of said genes. Creating strains in alternative diazotrophic microbes by making genetic modifications analogous to those described herein in the context of A. vinelandii is expected to produce genetically-modified strains that are phenotypically similar in relevant part to the strains expressly exemplified herein.

Preparation and Growth of A. vinelandii Strains

In one aspect, the present disclosure describes genetically modified diazotrophic microbes that produce γ-PGA. The diazotrophic microbe may be genetically modified to include genes encoding proteins, such as enzymes, that can synthesize γ-PGA. The diazotrophic microbe may be genetically modified to include elements to increase the amount of γ-PGA synthesized.

In one or more embodiments, the diazotrophic microbe described herein may be genetically modified to decrease expression of genes involved in a pathway, such as a metabolic pathway. This may decrease the function of the given pathway within the cell. The pathway may be known to use the same resources used in production of γ-PGA. Exemplary pathways may include, but are not limited to, lipid synthesis, complex carbohydrate synthesis, protein glycosylation, polyhydroxybutyrate production, or alginate production.

For example, the metabolic pathways used to produce alginate and/or polyhydroxybutyrate (PHB) may be knocked-down or knocked-out. FIG. 1B shows the structures of alginate and PHB, two biopolymers related to γ-PGA. Production of alginate and/or PHB could compete for carbon and metabolic resources with production of γ-PGA. Thus, decreased expression of the genes involved in these pathways may increase production of γ-PGA in the diazotrophic microbe, such as A. vinelandii.

In one or more embodiments, gene expression regulatory elements may be modified to achieve a desired level of gene expression. Regulatory elements include, but are not limited to, promoters, enhancers, ribosome binding sites (RBS), internal ribosome entry sites (IRES), polyadenylation signals, operators, repressors, recognition elements. In one or more embodiments, a gene may be optimized for the codon bias of a genetically modified microbe. In one or more embodiments, the diazotrophic microbe is engineered to utilize an optimized RBS. The operon sequences of two A. vinelandii strains with different RBS sequences are shown in Table 1. The RBS is shown in bold. The Shine-Dalgarno sequence is underlined. The start codon is shown in italics. The impact of modifying the RBS as shown in Table 1 is described in greater detail in EXAMPLE 3 herein.

Alternatively, in one or more embodiments, one may achieve a desired level of gene expression using a strategy that employs transposons that randomly insert exogenous genes for PHB production into A. vinelandii.

TABLE 1
RBS sites of pgsC and pgsA operons in
A vinelandii strain AZBB667 and AZBB697.

| Strain Name | Operon Sequence |
|---|---|
| AZBB667 | TAAGCTAGGGGGAAATGCAGACATGTTCGGA |
| | (SEQ ID NO: 1) |
| AZBB697 | TAAGCTAGTTAAGGAGGTAAAAAAAATGTTCGGA |
| | (SEQ ID NO: 2) |
| AZBB667 | TTAATTTAATGTAAGGTGTG TCAAACGATGAAAAAA |
| | (SEQ ID NO: 3) |
| AZBB697 | TTAATTTAATTAAGGAGGTAAAAAAAATGAAAAAA |
| | (SEQ ID NO: 4) |

In one or more embodiments, the diazotrophic microbe may include additional copies of genes involved in synthesis of γ-PGA. Genes considered to be involved in synthesis of γ-PGA may include, but are not limited to, genes involved in the creation of chemical energy, synthesis of cofactors, or synthesis or transport of precursors (e.g., glutamate). Additionally, or alternatively, the diazotrophic microbe may be modified to change gene expression elements associated with an existing metabolic pathway, such as increase the strength of an associated promoter. In one or more embodiments, the diazotrophic microbe includes genes associated with production of galactose. The diazotrophic microbe may include copies of genes associated with production of galactose in addition to any copies it may naturally have in its genome. Production of galactose may advantageously increase production of ammonia, ultimately increasing production of γ-PGA. In one or more embodiments, the presence of galactose moderates the production levels of the γ-PGA (e.g., in AZBB709 and AZBB726). Because γ-PGA production is modulated based on the level of galactose, one can use the presence of galactose to tune the level of γ-PGA production. In one or more embodiments, the diazotrophic microbe includes mutated copies of genes associated with the production of metabolites that limit production of γ-PGA.

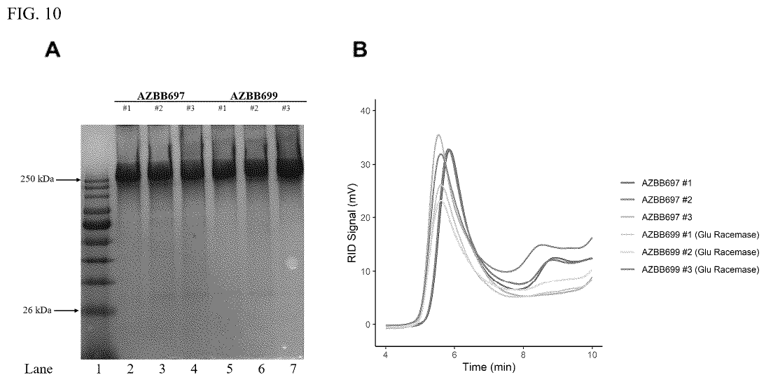

In one example, the B. subtilis glutamate racemase gene, racE, was placed behind the promoter of the S-layer (surface layer) protein (Avin_16040) in AZBB697. The S-layer protein promoter has been demonstrated to be a strong promoter. A. vinelandii possesses its own glutamate racemase, murI. A pairwise sequence alignment of racE and murI using EMBOSS Needle (ebi.ac.uk/Tools/psa/emboss_needle/) revealed that the two proteins have 41.8% sequence identity. Nonetheless, overexpression of glutamate racemase may lead to an increase in γ-PGA production if D-glutamate is the preferred glutamate enantiomer for A. vinelandii. Thus, A. vinelandii strain AZBB697 was modified to express racE under control of the S-layer promoter. This new strain was named AZBB699. The impact of this modification is described in EXAMPLE 5 and shown in FIG. 10.

All plasmids described herein were constructed and maintained within Escherichia coli JM109, which was obtained from New England Biolabs (Ipswich, MA). Methods for A. vinelandii genome editing have been previously described (Barney et al. 2015; Eberhart et al. 2016). Each strain of A. vinelandii prepared is described in Table 2.

TABLE 2
A. vinelandii strains tested for γ-PGA production

| Strain Name | Genetic Features |
|---|---|
| DJ | Wild-type A. vinelandii with decreased alginate production, adapted for |
| | plasmid transformation. |
| AZBB131 | ΔphbBAC |
| AZBB652 | ΔalgD.8.44.KJGXLIVFA |
| AZBB667 | ΔalgD.8.44.KJGXLIVFA |
| | phbB::pgsBCAE-Kan^{R} |
| AZBB690 | ΔalgD.8.44.KJGXLIVFA, phbB::LacZ-Kan^{R} |
| AZBB697 | ΔalgD.8.44.KJGXLIVFA |
| | phbB::pgsBCAE(B. subtilis 168, RBS optimized)-Kan^{R}) |
| AZBB699 | ΔalgD.8.44.KJGXLIVFA |
| | phbB::pgsBCAE(B. subtilis 168)-Kan^{R} |
| | Avin 16040::racE(B. subtilis 168, RBS optimized)-Strep^{R} |
| AZBB709 | pgsBCAE(Bacillus subtilis)-strep^{R}(behind Avin_51340 promoter) |
| | ΔalgD.8.44.KJGXLIVFA |
| AZBB710 | nasAB::pgsBCAE(Bacillus subtilis)-tet^{R}, ΔalgD.8.44.KJGXLIVFA |
| AZBB711 | Avin_16040::pgsBCAE(Bacillus subtilis)-strep^{R}, ΔalgD.8.44.KJGXLIVFA |
| AZBB725 | Avin_16040::pgsBCAE(Bacillus subtilis)-strep^{R}, |
| | ΔalgD.8.44.KJGXLIVFA, ΔphbBAC |
| AZBB726 | pgsBCAE(Bacillus subtilis)-strep^{R}(behind Avin_51340 promoter), |
| | ΔalgD.8.44.KJGXLIVFA, ΔphbBAC |
| AZBB725 | Disruption of Avin_22170 in AZBB725 background |
| Mut #4 | |

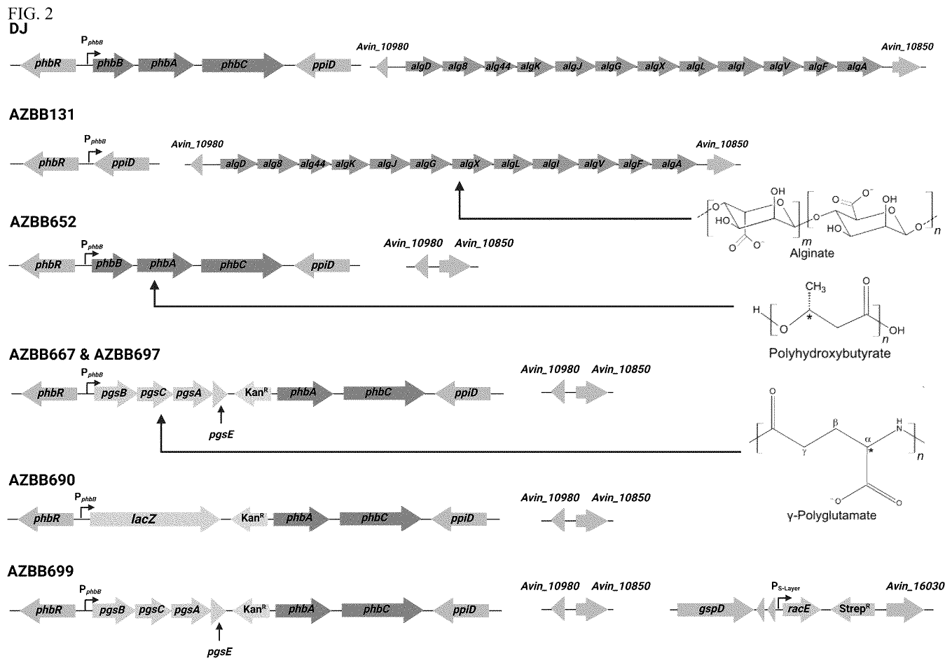

Strains AZBB667, AZBB690, AZBB697, and AZBB699 were kanamycin resistant, as indicated by “Kan^{R}”. Strain AZBB699 was streptomycin/spectinomycin resistant, as indicated by “Strep^{R}”. A summary of the genotypes of strains used in this work is depicted in FIG. 2.

Strain AZBB652, in which the genes for alginate synthesis were knocked out, was obtained. As the genes for alginate synthesis are knocked out, AZBB652 and strains derived therefrom should not produce alginate. Although alginate and PHB pathway knockdown is described herein, genetic manipulation of other pathways are also recognized as being likely to increase the expression of γ-PGA in a diazotrophic microbe.

A. vinelandii strain AZBB667 is derived from AZB652. AZBB667 expresses the polyglutamate synthase genes from B. subtilis 168 behind the native A. vinelandii phbB promoter and replaces phbB in the phbBAC operon, which synthesizes polyhydroxybutyrate (PHB). The phbB gene encodes an NADPH-dependent acetoacetyl-CoA reductase that reduces acetoacetyl-CoA. Downstream, (R)-β-hydroxybutyrate is polymerized by PHB synthase (phbC) to form PHB. Thus, AZBB667 should be deficient in both PHB production and alginate production.

The phbBAC operon produces polyhydroxybutyrate as well as additional polyhydroxyalkanoates (PHAs), such as polyhydroxyvalerate. In one or more embodiments, additional genes from the phbBAC operon are knocked down or replaced in the diazotrophic microbes described herein. In one or more certain embodiments, the entire phbBAC operon is replaced by another gene, such as a gene that synthesizes γ-PGA (Table 2).

In one or more embodiments, genes that synthesize γ-PGA, such as the pgsBCAE operon, may be inserted into any suitable location in the genome of a diazotrophic microbe. Suitable locations for gene insertion may be identified by a screening method, such as an insertional library screen. Alternatively, suitable locations for gene insertion may be identified by certain characteristics, such as proximal promoter strength, genomic accessibility to polymerases, or the role of the genes to be replaced. Inserted genes may replace one or more naturally existing genes, or they may be inserted without removal or replacement of any naturally existing genes.

### Lysis and Harvest of γ-PGA

Cells were grown according to previously described methods (Barney et al., 2015, Appl Environ Microbiol 81(13):4136-4328). Following the growth procedure, A. vinelandii cells were centrifuged for eight minutes at 4° C. and 6,200×gravity (g) and the supernatant was separated from the cell pellet. Supernatants were frozen overnight at −80° C. in Falcon tubes and lyophilized using a freeze dryer pulling vacuum at <0.12 mBar with a temperature of less than 43° C. until the water was removed. Samples were resuspended in deionized water to prepare an approximately 12.5-fold concentration of the supernatant. Periplasmic fractions of A. vinelandii were obtained using a method described previously (Barney et al. 2004).

Portions of the lyophilized samples were processed using a centrifugal filter device with a nominal molecular weight limit of 3 kDa. Up to 4 mL of sample was added to the filter device (500 μL at a time) and centrifuged at 14,000×g for 30 minutes. The filter device was then washed twice with 500 μL of deionized water. Filter devices were spun upside down at 5,000×g for three minutes and their contents were transferred to a pre-weighed collection tube. Samples were frozen at −80° C. and lyophilized using a freeze dryer pulling vacuum at <0.12 mBar with a temperature of less than 43° C. until the water was removed. The mass of each sample was calculated by weighing each tube after lyophilization.

While described herein in an exemplary embodiment in which the genetic modification involves modifying at least a portion of the A. vinelandii phbBAC operon, the genetically modified A. vinelandii strains and methods described herein can involve genetic modification of the A. vinelandii genome in other locations instead of, or in addition to, the exemplary modifications described herein. For example, the A. vinelandii strain may contain a genetic modification inserted as part of a transposon that randomly inserts the gene into many thousands of sites. The methylene blue plate screening method described herein may be used to find mutants that produce elevated levels of γ-PGA. This type of screen may enable screening of many mutant strains to identify more ideal sites for optimal expression.

### Analysis of γ-PGA Production Using PAGE

Gels used for Sodium dodecyl sulfate-polyacrylamide gel electrophoresis (SDS-PAGE) were prepared with a 10% acrylamide running portion and a 4% acrylamide stacking portion. For one gel, the 10% acrylamide gel included 1.25 mL of 1.5 M Tris-HCl, pH 8.8, 2.42 mL water, 25 μL 10% ammonium persulfate (APS), 50 μL 10% sodium dodecyl sulfate (SDS), 1.25 mL 40% acrylamide, and 5 μL tetramethylethylenediamine (TEMED). The 4% stacking gel included 625 μL of 0.5 M Tris-HCl, pH 6.8, 3.07 mL dH2O, 12.5 μL 10% APS, 25 μL 10% SDS, 300 μL 40% polyacrylamide, and 2.5 μL TEMED. Lyophilized supernatant samples were mixed with 2×Laemmli sample buffer that contained 5% β-mercaptoethanol. 15 μL volumes of sample were loaded onto the gel and run for 60 minutes at 165 V in a Bio-Rad electrophoresis box. Following electrophoresis, gels were stained for one hour with methylene blue dye (0.5% methylene blue, 3% acetic acid in water) and de-stained using water until the dye was sufficiently removed from the gel and bands were visible.

An SDS-PAGE gel containing 3 mg mL-1 dry weight of lyophilized and ultrafiltered supernatant samples prepared as described in Sections 1.1.3 and 1.1.4 and as well as standards of γ-PGA (0.1, 0.25, 0.5, 1 mg mL-1) was analyzed for densitometry using the ImageJ software package (https://imagej.nih.gov/ij/download.html). The relative densities of the γ-PGA standards and experimental samples were measured using a rectangle of a given area. The densities of four regions of the gel with no bands were taken and averaged to yield a blank. The blank was subtracted from all other density measurements. This process was done in triplicate three times and the three replicates were summed to yield the final cumulative densities. A standard curve was developed from the γ-PGA standard cumulative densities, and the concentration of the experimental samples was estimated by applying the standard curve equation to the cumulative densities of the experimental samples.

### Analysis of γ-PGA Production Using Size Exclusion Chromatography

Lyophilized supernatant samples were diluted in 10 mM sodium citrate buffer and filtered through a 0.22 μm syringe filter (Tisch Environmental, Inc). 20 μL of filtered sample was injected into a Shimadzu LC-10AT High Performance Liquid Chromatograph System fitted with a Sepax SRT SEC-300 size exclusion chromatography column (4.6 mm×300 mm column dimension, 4.98 mL column volume, 300 Å pore size) with a column oven temperature of 50° C. and a flow rate of 0.35 mL/min. Samples were subsequently processed with a Shimadzu RID-10A Refractive Index Detector and the chromatography data analyzed using Clarity Lite Chromatography Software (https://www.dataapex.com/download).

### High-Performance Liquid Chromatography (HPLC)

Portions of lyophilized and ultrafiltered supernatant samples were digested with 6 N HCl at 110° C. for 24 hours. Following digestion, the samples were centrifuged, and the supernatant of each sample was diluted 10 times with water and filtered with a 0.22-μm syringe filter. The filtrate was derivatized and analyzed with high performance liquid chromatography using a derivation of a method previously described (Henderson and Brooks 2010).

### Nuclear Magnetic Resonance (NMR)

Portions of lyophilized and ultrafiltered supernatant samples prepared as described herein were subject to proton nuclear magnetic resonance (1^{H}-NMR) using a Varian Unity 400 MHz Spectrophotometer. 1^{H}-NMR spectra were obtained with 64 scans and a 5.5 ms pulse width. Deuterium oxide (D₂O) was used as a solvent and the data was processed using MestReNova software (https://mestrelab.com/download/mnova/).

### Transposon Mutagenesis

A. vinelandii strain AZBB667 was streaked twice from a 7% DMSO frozen stock onto B medium plates supplemented with 3 μg/mL kanamycin and incubated at 30° C. for three days each time. Escherichia coli strain WM3064 containing plasmid pBB298 was streaked twice from a frozen stock on LB plates supplemented with 15 μg/mL tetracycline and 100 μL of a 5 mg/mL stock of diaminopimelic acid (DAP) and incubated at 30° C. for one day each time. A full inoculation loop of A. vinelandii cells were resuspended in 500 μL dH2O and a full inoculation loop of E. coli cells were resuspended in 500 μL LB. E. coli and A. vinelandii were combined in ratios of 1:10 (20 μL: 200 μL) and 1:20 (10:200 μL) and ten dots of each were plated on standard Burk's medium plates plus 5 g/L yeast extract and supplemented with 100 μL of a 5 mg/mL stock of DAP. Plates were incubated right-side up at 30° C. overnight.

The following day, the ten spots on each plate were combined into two groups of five and inoculated into 50 mL of Burk's medium in base/acid washed 125 mL Erlenmeyer flasks. Cultures were grown at 28° C. with agitation at 180 rpm overnight. The following day, 0.5 mL and 2 mL were drawn from the cultures and were centrifuged at 10,000×g, and the cells were resuspended in 100 μL of supernatant. These four-fold dilutions were each plated on a B medium plate supplemented with 15 μg/mL of tetracycline and 20 mg/L of methylene blue dye. Plates were monitored daily for one week to assess colony phenotypes. Colonies that were selected were streaked twice to B medium plates with tetracycline and methylene blue and then streaked a final time to B medium plates with just tetracycline. All plates were incubated at 30° C. for three days. Cells were scraped from plates and genomic DNA was extracted using a kit (Zymo Research, Catalog #D6005, Irvine, CA). Genomic DNA was digested with PstI and cleaned and concentrated (Zymo Research, Irvine, CA). Digested gDNA was ligated with T4 ligase (New England Biolabs, Ipswich, MA) and cleaned and concentrated. Ligations with the tetracycline transposon insertion were amplified using primers BBP1175 and BBP1176 and sequenced with BBP3118 using Sanger sequencing (ACGT DNA Sequencing Services, Wheeling, IL).

Preparation and Growth of A. vinelandii Strains Using Carbon Sources Found in Root Exudates.

In another aspect, the present disclosure describes a genetically modified diazotrophic microbe that can produce ammonium when grown on a carbon source associated with central metabolism. In particular, the present disclosure describes a genetically modified diazotrophic microbe that can utilize tricarboxylic acid (TCA) cycle intermediates as a primary carbon source.

Beneficial plant-microbe interactions are typically reliant on the establishment of bacterial strains in the rhizosphere of the plant. This interaction is influenced by many factors, one of which is root exudates. Plants release between 5-25% of net fixed carbon into the rhizosphere in the form of small organic molecules like sugars, amino acids, and organic acids that have a significant influence on soil properties, the microbial community, and soil functions. Sugars can constitute 70% of total root exudate carbon. Glucose, sucrose, arabinose, xylose, fructose, and galactose have been identified in exudates of Arabidopsis spp., rice, maize, wheat, and barley. Organic acids like citrate, malate, succinate, oxalate, and fumarate are the most commonly released organic acids from plants and their secretion constitutes an important strategy used by plants to cope with the limitation of nutrients such as iron, phosphorus, potassium, nitrogen, and zinc. Biological nitrogen fixation in A. vinelandii is regulated by the nifLA operon. Expression of NifLA is constitutive and does not depend on nitrogen levels. NifA is an activator that must bind upstream of the promoters of all the nif operons to enable their expression whereas NifL is an inhibitor that binds to NifA in a 1:1 ratio and neutralizes it in the presence of oxygen or ammonium.

The NifA protein is composed of an N-terminal regulatory domain (GAF), a central AAA+ sigma-54 activation domain, and a C-terminal DNA-binding domain (Batista et al. 2021). The GAF regulatory domain of NifA regulates the interaction with NifL in response to the concentration of 2-oxoglutarate (2-OG) which binds directly to the GAF domain. Thus, 2-OG is considered a “master regulator” due to its involvement in many regulatory pathways and its importance in carbon and nitrogen metabolism (Huergo and Dixon 2015). Under excess carbon (reducing) and limiting nitrogen conditions, 2-OG binds to the GAF regulatory domain of NifA, which allows it to escape regulation from NifL by inducing a conformational change (Little and Dixon 2003). This ultimately results in increased nitrogen fixation.

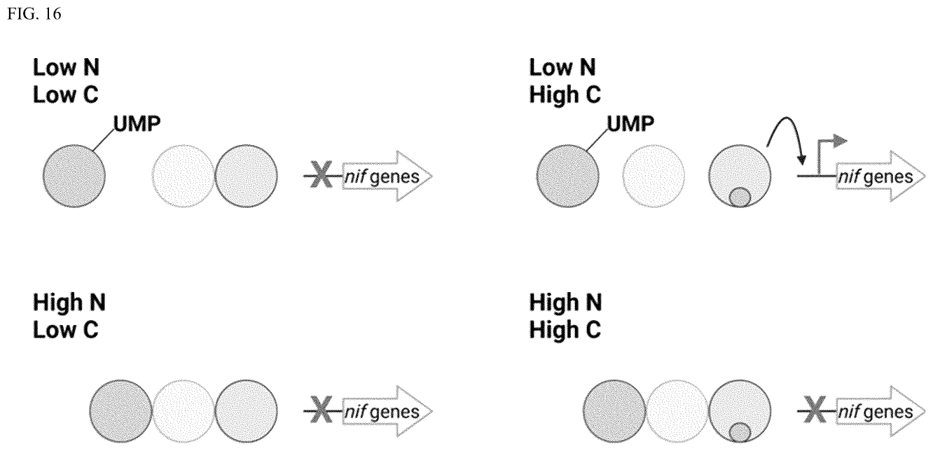

Regulation of nif genes is also dependent on PII proteins which control the activity of NifA (Huergo and Dixon 2015). GlnK is a PII protein in A. vinelandii that modulates the interaction between NifL and NifA in response to nitrogen levels. The activity of GlnK is dependent on its uridylylation status. Glutamine is a nitrogen signaling molecule that can indicate nitrogen status since glutamine synthetase synthesizes glutamine from glutamate and ammonia (Huergo and Dixon 2015). GlnK is uridylylated by the uridylyl transferase/uridylyl-removing enzyme GlnD, which is allosterically regulated by glutamine (Jiang et al. 1998). When glutamine levels are high, meaning there is excess nitrogen, GlnK is deuridylylated and interacts with NifL, forming a GlnK-NifL-NifA ternary complex that inactivates NifA. Even under excess carbon conditions when 2-OG levels are high and 2-OG is bound to NifA, the deuridylylated GlnK renders 2-OG unable to prevent NifL from binding to NifA, thus inhibiting it (Little and Dixon 2003). In contrast, when glutamine levels are low, GlnK is uridylylated and does not interact with NifL. However, under carbon limiting (oxidative) conditions the FAD of NifL is oxidized and NifL inhibits transcriptional activation of NifA (Huergo and Dixon 2015; Little et al. 2002; Hill et al. 1996). This response to redox status prevents the expression of nif genes at oxygen levels that could result in oxidative inactivation of nitrogenase (Mus et al. 2022). Only when NifL is reduced does it not inhibit NifA. Thus, 2-OG is an effective allosteric regulator only under reducing (excess carbon) and nitrogen-limiting conditions which allow NifA to activate transcription of nif genes (Huergo and Dixon 2015; Little et al. 2002). A schematic is shown in FIG. 16.

Efforts to boost ammonia production in A. vinelandii through nifL disruptions have been explored. However, deleting nifL is not sufficient to result in increased ammonium secretion. Therefore, alternative mechanisms of nifLA regulation may exist in A. vinelandii.

It has been suggested that a high ammonium excreting phenotype was possible when nifL deletions were accompanied by increased expression of rnf1, thus possibly increasing the availability of reducing equivalents to support nitrogenase activity. These alterations did not increase ammonium secreted by themselves.

Thus, moderating the balance of 2-OG, related metabolic intermediates, and reducing equivalent availability may impact the level of ammonium secreted by A. vinelandii. This disclosure describes, in part, methods of genetically engineering diazotrophic microbes to improve utilization of metabolic intermediates as a primary carbon source during diazotrophic growth.

TABLE 3
A. vinelandii strains tested for ammonium production in multiple carbon sources.

| Strain Name | Genetic Features |
|---|---|
| AZBB163 | nifL::kanR-(pPCRNH3-43) |
| AZBB568 | Avin_11360-Kan^{R}-pPCRNH3-44-nifA-Avin_11370, ΔnifLA |
| AZBB689 | nifL::Kan^{R}-pPCRNH3-44, ΔrnfABCDGEH, fixABCX::tet^{R}- |
| | Avin_16040::fixABCX-strep^{R}(fix RBS site) |
| AZBB695 | rnfABCDGEH::tet^{R}, Avin_11360-Kan^{R}_pPCRNH3-44-nifA-Avin_11370, ΔnifLA |
| AZBB696 | fixABCX::tet^{R}, Avin_11360-kan^{R}_pPCRNH3-44-nifA-Avin_11370, ΔnifLA |

In one aspect, the present disclosure relates to an engineered diazotrophic microbe that uses non-standard carbon sources to grow. In one or more embodiments, the engineered diazotrophic microbes use non-standard carbon sources to produce ammonium.

As it is used herein, a non-standard carbon source refers to any source of carbon not commonly used for in vitro growth of microbes, such as sucrose. In particular, the present disclosure relates to non-standard carbon sources commonly found in root exudate. In one or more embodiments, the non-standard carbon source includes a sugar such as glucose, fructose, ribose, xylose, arabinose. In one or more embodiments, the non-standard carbon source includes a primary organic acid such as citrate, malate, and succinate.

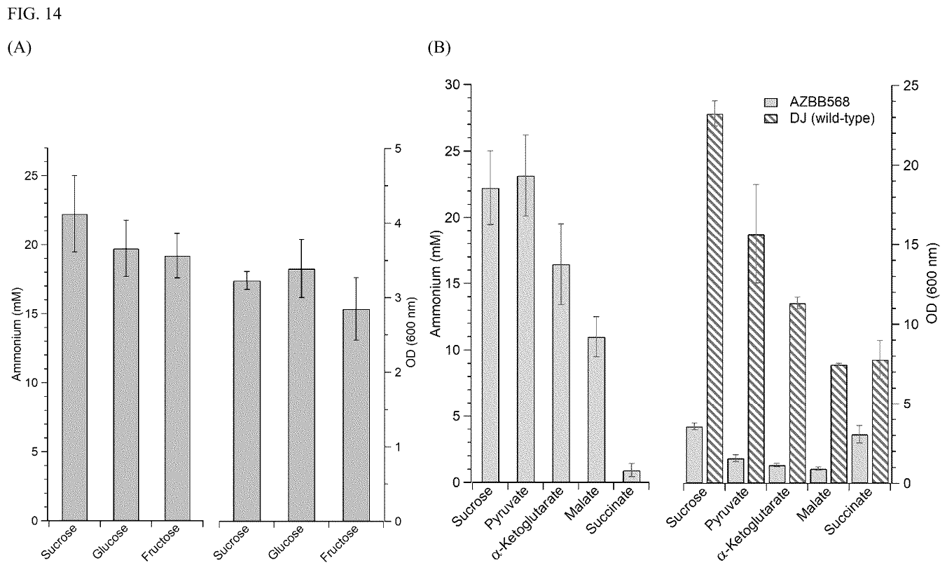

AZBB568 grew to a similar cell density when provided with sucrose, glucose, or fructose. FIG. 14A). Ammonium production with the sugars sucrose, glucose and fructose were all similar for AZBB568 (FIG. 14A), while ribose, arabinose and xylose did not result in any substantial growth for AZBB568 or wild-type A. vinelandii (data not shown). In one or more embodiments, an engineered diazotrophic microbe uses glucose and/or fructose to grow. In one or more embodiments, an engineered diazotrophic microbe grown on media lacking sucrose but including glucose and/or fructose grows more quickly than a non-engineered diazotrophic microbe grown on media lacking sucrose but including glucose and/or fructose. In one or more embodiments, an engineered diazotrophic microbe uses glucose and/or fructose to produce ammonium. In one or more embodiments, an engineered diazotrophic microbe grown on media lacking sucrose but including glucose and/or fructose produces more ammonium than a non-engineered diazotrophic microbe grown on media lacking sucrose but including glucose and/or fructose.

Organic acids resulted in a much higher degree of variation depending on the specific organic acid selected (FIG. 14B). No growth was observed with citrate, oxaloacetate, and fumarate, even for wild-type A. vinelandii. The organic acids malate and succinate are primary components of the TCA cycle and are also common to root exudates, and these organic acids did result in measurable growth of both wild-type A. vinelandii and the AZBB568 strain. While strains were able to grow on succinate and malate, the cell densities that were obtained were lower than what was found for various sugars (approximately one third of the OD). A large difference was found for ammonium produced between succinate and malate (FIG. 14B). While malate achieved ammonium levels near 10 mM, succinate only reached about 1 mM. Additionally, the organic acids pyruvate and α-ketoglutarate both resulted in higher levels of ammonium accumulated versus malate, with pyruvate achieving ammonium levels comparable to what was found with sucrose (FIG. 14B).

In one or more embodiments, an engineered diazotrophic microbe uses malate, succinate, and/or α-ketoglutarate to grow. In one or more embodiments, an engineered diazotrophic microbe grown on media lacking sucrose but including malate, succinate, and/or α-ketoglutarate grows more quickly than a non-engineered diazotrophic microbe grown on media lacking sucrose but including malate, succinate, and/or α-ketoglutarate. In one or more embodiments, an engineered diazotrophic microbe uses malate, succinate, and/or α-ketoglutarate to produce ammonium. In one or more embodiments, an engineered diazotrophic microbe grown on media lacking sucrose but including malate, succinate, and/or α-ketoglutarate produces more ammonium than a non-engineered diazotrophic microbe grown on media lacking sucrose but including malate, succinate, and/or α-ketoglutarate.

In the preceding description, particular embodiments may be described in isolation for clarity. Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

For any method disclosed herein that includes discrete steps, the steps may be performed in any feasible order. And, as appropriate, any combination of two or more steps may be performed simultaneously.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

### EXAMPLES

The present invention is illustrated by the following examples. It is to be understood that the particular examples, materials, amounts, and procedures are to be interpreted broadly in accordance with the scope and spirit of the invention as set forth herein.

### Example 1

In this Example, production of γ-PGA in genetically engineered A. vinelandii strains was qualitatively characterized using SDS-PAGE and size exclusion chromatography.

### SDS-PAGE Analysis

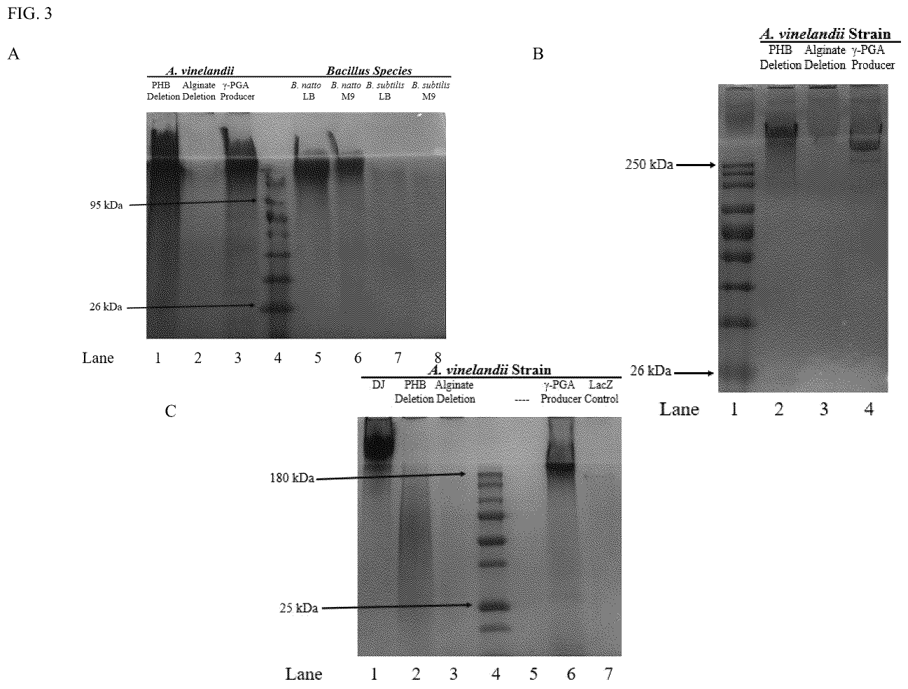

AZBB131, AZBB667, AZBB652, B. natto, and B. subtilis 168 were grown as described. B. natto and B. subtilis 168 were grown in either LB medium or M9 medium. Supernatant from each culture was collected and run on an SDS-PAGE gel. The gel was stained using methylene blue, and is shown in FIG. 3A. AZBB131 (Lane 1) and AZBB667 (Lane 3) both display bands that indicate high molecular weight polymers. Presumably, the band for AZBB131 is alginate while the band for AZBB667 is γ-PGA. This is bolstered by the fact that AZBB652 (Lane 2), which is deficient in alginate production, does not show any type of band. It is important to note that AZBB652 does produce PHB and that the lack of a band from PHB should also be expected since it does not have a negatively charged side group (FIG. 1B). Furthermore, B. natto, which produces γ-PGA, but not alginate or PHB, also shows a high molecular weight band (Lanes 5-6) while B. subtilis 168, which is unable to produce γ-PGA, does not display any bands (Lanes 7-8).

Since A. vinelandii is a Gram-negative bacterium, γ-PGA could have been trapped in the periplasm of the organism since heterologous expression of pgsBCA in E. coli led to such low yields (Ashiuchi et al. 1999; Cao et al. 2013; Liu et al. 2020). The periplasmic fractions of AZBB131, AZBB652, and AZBB667 cells grown for seven days were extracted and stained with methylene blue on an SDS-PAGE gel as well. As shown in FIG. 3B, some γ-PGA does appear to be in the periplasmic space (Lane 4), as is the case with the extracellular alginate in AZBB131 (Lane 2).

AZBB667 was further tested against two other control strains, A. vinelandii DJ (wild-type) and AZBB690, which has a non-alginate producing phenotype with lacZ, an innocuous gene, inserted in place of phbB. As shown in FIG. 3C, methylene blue staining of the supernatants revealed no band for the AZBB690 (Lane 7) control as expected while DJ (Lane 1) exhibited a dark band of very high molecular weight which is presumably alginate.

### Size Exclusion Chromatography

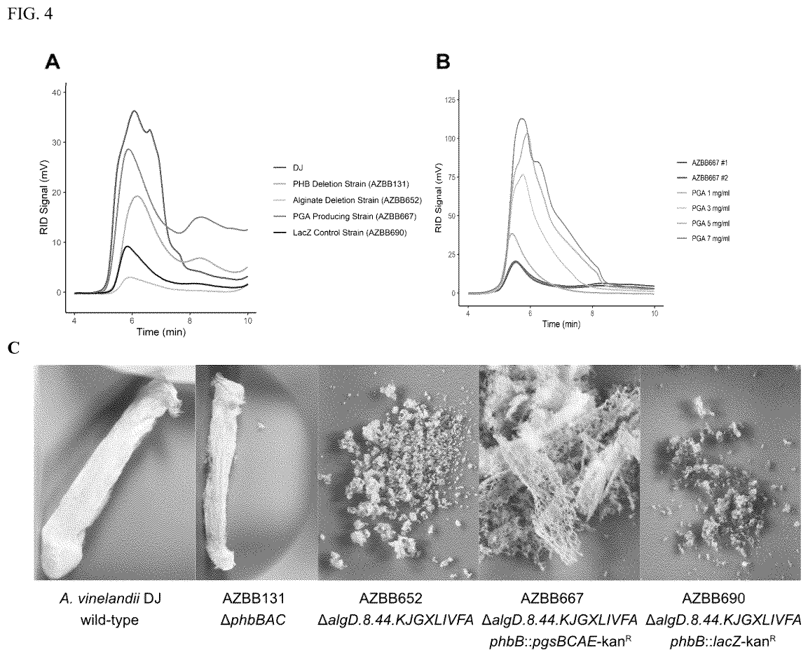

Further analysis by size exclusion chromatography (SEC) revealed some intriguing results. Among the strains that are alginate deficient, AZBB667 produced a larger peak around a retention time of six minutes than did AZBB652 and AZBB690, which cannot produce γ-PGA (FIG. 4A). This could indicate the presence of a greater amount of total high molecular weight polymer in the sample. It is unknown what exactly is responsible for the much smaller peaks attributed to AZBB652 and AZBB690, but other high molecular weight compounds in the cell could possibly account for this. FIG. 4B shows SEC results comparing AZBB667 to standards of purchased γ-PGA that was extracted from Bacillus natto. While those results do not prove by itself that the AZBB667 peak is γ-PGA, it does show that γ-PGA has a retention time of approximately six minutes using the Sepax SRT SEC-300 column. According to its user manual, the Sepax SRT SEC-300 column elutes compounds with a molecular weight of ˜670 kDa at ˜7 minutes and the molecular weight of γ-PGA can range from 100-1,000 kDa. Since larger compounds elute faster in SEC, a peak around six minutes could indicate that the γ-PGA from AZBB667 is larger than 670 kDa.

The results of the PAGE gel shown in FIG. 3C and the chromatograms shown in FIG. 4A and FIG. 4B qualitatively suggest that γ-PGA is produced in A. vinelandii strain AZBB667.

### Visual Analysis

Another qualitative analysis is a visual comparison of the lyophilized supernatants of the different A. vinelandii strains. FIG. 4C shows what appears to be a thick polymer for both alginate producing strains, A. vinelandii DJ and AZBB131, while the two alginate deletion strains, AZBB652 and AZBB690, show what looks like leftover media components including siderophores produced by the Azotobacter. Conversely, the γ-PGA producer, AZBB667, appears to show what could be some type of polymer too as evidenced by a sponge-like matrix that is markedly different from those of A. vinelandii DJ and AZBB131.

### Example 2

In this Example, samples of the supernatants collected in EXAMPLE 1 were filtered and subject to quantitative analysis to measure the presence of γ-PGA.

### Ultrafiltration

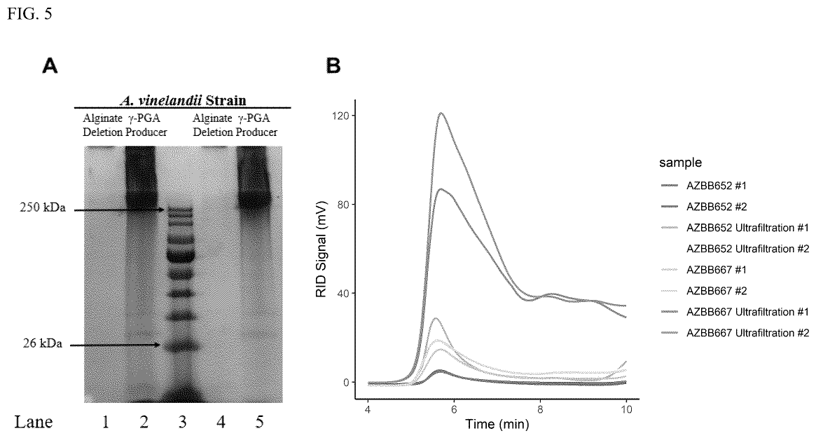

To further characterize the supernatants of EXAMPLE 1, extracellular levels of glutamate were measured. Following the compelling evidence that AZBB667 is able to produce γ-PGA amino acid analysis was performed to check if extracellular levels of glutamate were higher for AZBB667 as compared to AZBB652 and A. vinelandii DJ. For this experiment 250 mL cultures were grown and the lyophilized supernatants were resuspended in 20 mL dH2O, constituting a 12.5× concentration from the original volume. 4 mL of these concentrated supernatants were subjected to ultrafiltration and lyophilized as described herein. The dried concentrates were resuspended in 500 μL dH2O, constituting a further 8× concentration. The concentrates were analyzed via methylene blue staining of an SDS-PAGE gel (FIG. 5A) and size exclusion chromatography to ensure recovery of the polymer (FIG. 5B). FIG. 5A shows that the concentrate from the γ-PGA producer AZBB667 still displays a very dark band of high molecular weight (Lanes 2 and 5) while its parent strain, AZBB652, displays no band (Lanes 1 and 4). The AZBB667 bands in FIG. 5A appear much darker than the AZBB667 bands in FIG. 3, indicating a more concentrated polymer. This is further supported by FIG. 5B, in which the peaks of the AZBB667 samples that were filtered are significantly larger than the AZBB667 peaks that were not filtrated. The larger peaks for the filtrated samples are due to the 8× concentration. In essence, these results demonstrate a product recovery following ultrafiltration.

Supernatants that had been subject to ultrafiltration and lyophilized were subject to amino acid analysis as described herein. Table 3 displays the glutamate profile of A. vinelandii DJ, AZBB652, and AZBB667. From 250 mL cultures, the dry mass of the AZBB667 supernatant following lyophilization was approximately 3.7-fold greater than that of AZBB652 and approximately 2.7-fold greater than that of A. vinelandii DJ. Amino acid analysis following ultrafiltration showed that about 9-10% of the dry mass was glutamate whereas about 1-2% of the dry mass of DJ and AZBB652 was glutamate. However, approximately 22-30% of the dry mass of AZBB667 was reported as amino acids whereas only 4-7% of the dry mass of DJ and AZBB652 was amino acids. Nonetheless, glutamate still constituted approximately 34-40% of the total amino acids present in AZBB667 which was a noticeable increase over DJ and AZBB652, for which glutamate represented only about a quarter of the total amino acid profile.

TABLE 3
Glutamate profile of A. vinelandii strains

| | | Dry | Total Amino | Glutamate | Glutamate % |
|---|---|---|---|---|---|
| Strain | | mass | Acids | (mg/g | of total |
| name | Replicate | (mg) | (mg/g DM) | DM) | AA |
| DJ | 1 | 525.3 | 73.57 | 19.84 | 26.97 |
| AZBB652 | 1 | 362.9 | 43.13 | 10.41 | 24.14 |
| AZBB652 | 2 | 393.5 | 53.16 | 12.04 | 22.65 |
| AZBB667 | 1 | 1300.1 | 300.68 | 104.18 | 35.65 |
| AZBB667 | 2 | 1507.4 | 224.66 | 90.82 | 40.43 |

### Proton Nuclear Magnetic Resonance Spectroscopy

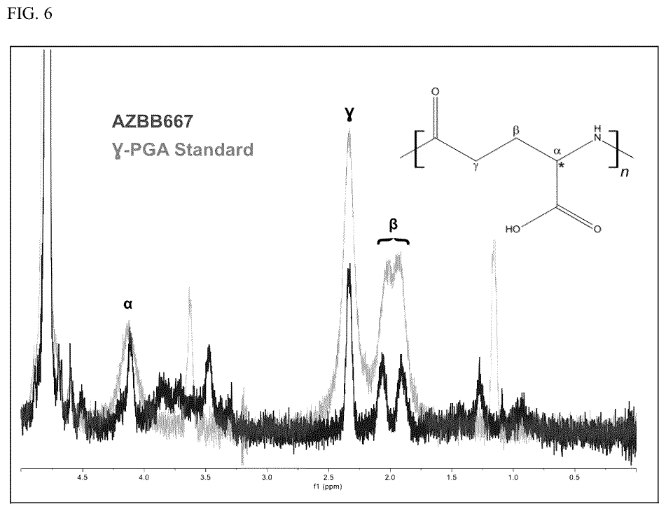

H¹-NMR was used to determine if extracellular γ-PGA was present by comparing ultrafiltrate supernatant with a known standard of γ-PGA. As shown in FIG. 6, the spectra overlap with the proton of the α-carbon displaying a peak at 4.1 ppm, the protons of the γ-carbon displaying a peak at 2.3 ppm, and the protons of the β-carbon displaying a split peak at 2.1 and 1.9 ppm. Peaks for AZBB667 between 3.5-4.0 ppm and 1.0-1.5 ppm can be attributed to impurities in the sample, most likely other amino acids (Khalil et al. 2016). The peaks at 1.2 and 3.6 ppm for the γ-PGA standard resemble the H1-NMR spectrum for ethanol, which was most likely the organic solvent used to extract the purified γ-PGA (Gerothanassis et al. 2002). The spectra for AZBB667 also agree with H1-NMR spectra of γ-PGA that have been previously reported (Khalil et al. 2016; Matsusaki et al. 2022; Peng et al. 2010).

From this Example, quantitative data confirmed that γ-PGA was produced by A. vinelandii strain AZBB667.

### Example 3

In this Example, a strain of A. vinelandii with an optimized ribosomal binding sequence (RBS) used to enhance production of γ-PGA by A. vinelandii strains.

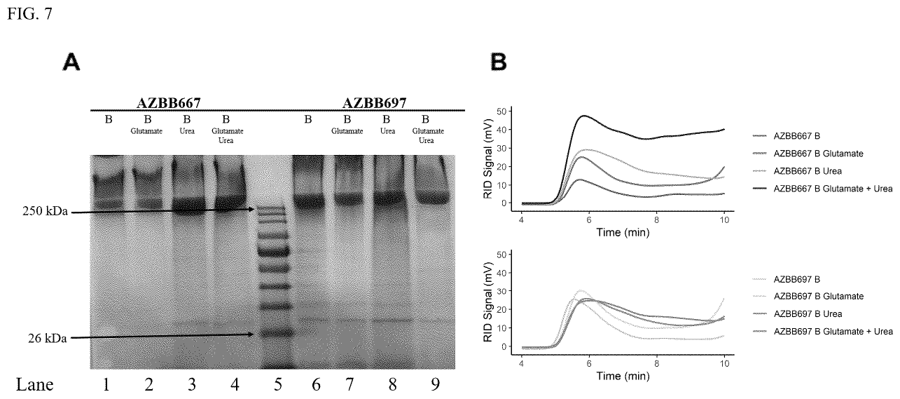

Strains AZBB667 and AZBB697 were grown in 250 mL cultures under different experimental conditions: B medium with no modifications, B medium with 2 g/L of L-glutamate supplemented, B medium with 5 mM urea, and B medium with 2 g/L of L-glutamate and 5 mM urea. Previous data indicate that AZBB667 can produce a small amount of γ-PGA in the absence of exogenous γ-PGA. Supplementing the media with 2 g/L of L-glutamate provides the strains with an exogenous source of L-glutamate that could possibly augment γ-PGA production while urea provides an exogenous nitrogen source that can be used in many cellular functions including amino acid biosynthesis and, thus, represses nitrogenase activity and allows for metabolic resources to be directed to other purposes. A sample of the data is shown in FIG. 7. The addition of L-glutamate did not seem to have an impact on γ-PGA production while the addition of urea may have had a marginal impact on AZBB667.

Subsequent amino acid analysis of AZBB667 and AZBB697 revealed no significant change in glutamate content with regards to both RBS optimization of the pgs operon as well as whether the medium was supplemented with an exogenous source of L-glutamate (Table 4). Glutamate was still approximately 9-14% of the total dry mass of the concentrate following ultrafiltration and roughly a third of the total amino acid profile.

TABLE 4
Glutamate profile of A. vinelandii
strains AZBB667 and AZBB697.

| | | Dry | Total Amino | Glutamate | Glutamate % |
|---|---|---|---|---|---|
| Strain | Growth | mass | Acids | (mg/g | of total |
| name | Conditions | (mg) | (mg/g DM) | DM) | AA |
| AZBB667 | B | 1985.5 | 459.49 | 133.26 | 29.00 |
| AZBB667 | B + Glu | 2100.25 | 286.72 | 92.80 | 32.37 |
| AZBB697 | B | 2614.25 | 356.72 | 112.56 | 31.55 |
| AZBB697 | B + Glu | 2423.25 | 394.55 | 145.10 | 36.78 |

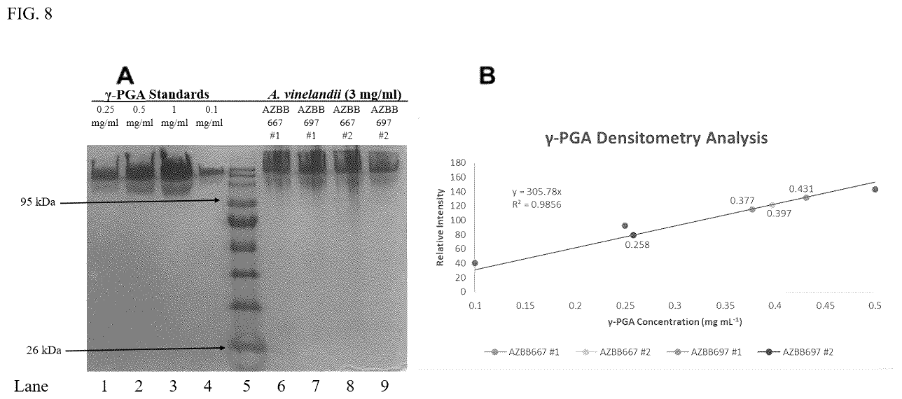

Densitometry analysis was used to estimate the yields of γ-PGA from A. vinelandii strains ABZZ667 and AZBB697. Ultrafiltered, concentrated samples of AZBB667 and AZBB697 lysate were run on an SDS-PAGE gel and stained with methylene blue. A standard curve using γ-PGA standards ranging from 0.1 to 1 mg/mL was used to quantify the concentration of the lysate samples. Only 0.1, 0.25, and 0.5 mg/mL standards were used for the calculation of the standard curve, as the gel is visually saturated 0.5 mg/mL. Density measurements of AZBB667 and AZBB697 were taken and applied to the standard curve equation (y=305.78x, R2=0.9856) to calculate the concentration of γ-PGA. The results suggest that the RBS-optimized strain AZBB697 does not produce higher amounts of γ-PGA than AZBB667 (FIG. 8B).

### Example 4

In this Example, a transposon was used to mutate the A. vinelandii strain AZBB667 to identify genes associated with γ-PGA synthesis.

Strain AZBB667 was transformed with E. coli strain WM3064 that donated a tetracycline transposon cassette from the plasmid pBB298 (FIG. 10A) for random insertion in the genome as described herein. Colonies were plated on B medium with 0.02 g/L of methylene blue and visualized under a microscope. Fourteen colonies with interesting phenotypes including size, morphology, color, and methylene blue dye pattern were selected from thousands and streaked on new B medium plates with methylene blue. Six of the colonies did not grow. Eight colonies grew and were prepared for sequencing. Sequencing revealed that the transposon inserted into the A. vinelandii genome as shown in Table 5.

TABLE 5
AZBB667 Transposon-Generated Mutants

| | Insertion Site | Gene Product | Potentially affected |
|---|---|---|---|
| Colony No. | Insertion direction | (Gene name) | Proximal Genes |
| Colony 5 | Avin_49970 (756 nt) | Enolase | Avin_49960 |
| | Forward, 37 nt from end | | tpiA-1 |
| Colony 6 | Avin_25210 (1776 nt) | Acyl-CoA | Avin_60570 |
| | Forward, 182 nt from end | dehydrogenase | tRNA-Cys |
| | | | Avin_60680 |
| | | | tRNA-Leu |
| Colony 9 | Avin_45910 (5464 nt) | Alkyl hydroperoxidase | Avin_45890 |
| | Reverse, 92 nt from end | reductase (ahpC) | ntrB |
| | | | Avin_45900 |
| | | | ntrC |
| Colony 10 | Avin_21190 (3993 nt) | Enterochelin synthase | Avin_21200 |
| | Reverse, 2161 nt from end | component F | entB |
| | | (entF) | Avin_21210 |
| | | | entE |
| Colony 11 | Avin_01070 (330 nt) | Hypothetical | Avin_01080 |
| | Forward, 292 nt upstream | Protein | Conserved Hyp. |
| | | | Avin_01090 |
| | | | Oliopeptidase A |
| Colony 12 | Avin_48590 (2289 nt) | Phosphate ABC | Avin_48580 (pstA) |
| | Forward, 971 nt from end | Transporter | Avin_48570 (pstB) |
| Colony 13 | Avin_43200 (831 nt) | Molybdenum storage | Avin_43210 (mosB) |
| | Reverse, 282 nt from end | protein sub. A | |
| Colony 14 | psgB (1182 nt) | Polyglutamate synthase | Avin_23660 (phbR) |
| | Reverse, 877 nt from end | subunit B (psgB) | Avin_23670 (phbH) |
| | | | Avin_23680 (phbF) |

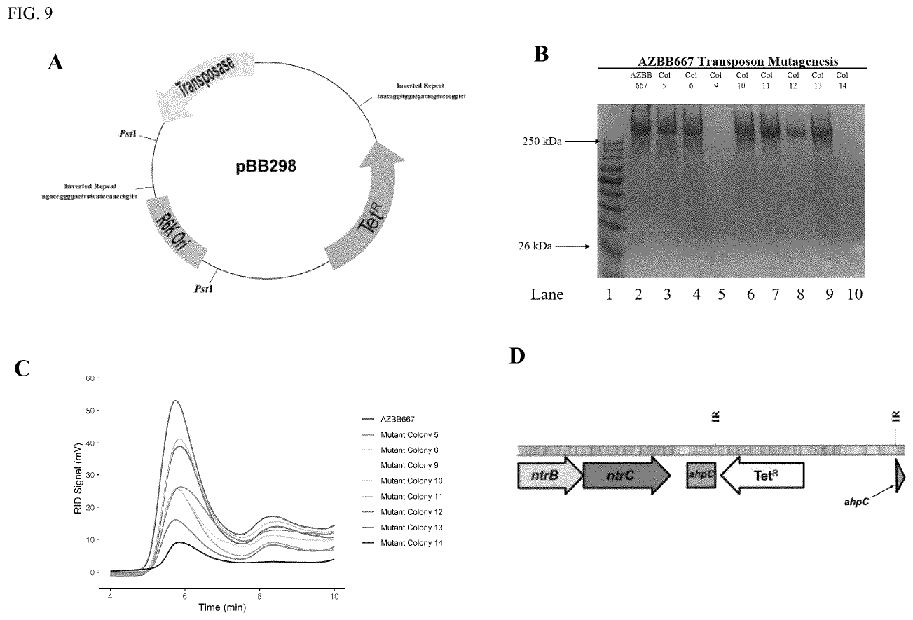

Supernatant from each mutant was analyzed using SDS-PAGE (FIG. 9B). Colonies 5, 6, 10, 11, and 13 showed similar patterns to the parent AZBB667 control when stained with methylene blue. Colonies 9 and 14 showed almost complete loss of product. Colony 12 showed some loss of product.

Supernatant from each sample was analyzed using size-exclusion chromatography and compared to supernatant from the parent AZBB667 strain as a control. Each mutant colony had a lower signal than AZBB667. Consistently with SDS-PAGE analysis, colonies 9 and 14 had the lowest signal, followed by colony 12.

Interestingly, sequencing of Colony 14 revealed that the transposon had been inserted into pgsB, one of the catalytic subunits of γ-PGA synthase. This, combined with the near-complete loss of product signal, suggests that the product detected in AZBB667 supernatant is, in fact, γ-PGA. The loss of product observed in Colony 9 suggest that alkyl hydroperoxidase reductase may additionally play a role in synthesis of γ-PGA.

In Colony 9 the transposon inserted into an alkyl hydroperoxide reductase, ahpC, and could potentially disrupt expression of two nearby genes, ntrB and ntrC, that are involved in the regulation of glutamine synthetase. A schematic of this is genomic region is shown in FIG. 9D.

### Example 5

In this Example, the AZBB699 strain of A. vinelandii, which includes a copy of B. subtilis glutamate racemase gene racE was assayed for expression of γ-PGA.

Results are shown in FIG. 10 and indicate that expression of racE behind the S-layer promoter did not lead to an increase in γ-PGA production.

### Example 6

In this Example, a strain of A. vinelandii, which includes a copy of B. subtilis glutamate racemase gene racE under control of the glutamate promoter was assayed for expression of γ-PGA.

Several additional strains were constructed (Table 6), and quantified the amounts of additional extracellular polymers (dry weight) that were obtained as a result. Three new strains (AZBB725, AZBB726, and AZBB725 Mut #4 (containing a mutation in gene Avin_22170)) have yielded significant increases in extracellular polymer levels (versus levels Alex found for his primary strain, AZBB667). Strain AZBB725 Mut #4 (containing the disruption in Avin_22170) also about 350 mg/L of extracellular polymer, which is almost nine-fold higher than the 40 mg/L observed for strain AZBB667.

TABLE 6

| | | Methylene | PGA |
|---|---|---|---|
| Strain | Genotype | Blue | quantification |
| DJ | Wild type | Minimal | N/A |
| AZBB652 | ΔalgD.8.44.KJGXLIVFA | | |
| AZBB667 | phbB::pgsBCAE(Bacillus subtilis)- | | 40 mg/L |
| | kan^{R}, ΔalgD.8.44.KJGXLIVFA | | |
| AZBB709 | pgsBCAE(Bacillus subtilis)-strep^{R}(behind | Increased | |
| | Avin_51340 promoter) | | |
| | ΔalgD.8.44.KJGXLIVFA | | |
| AZBB710 | nasAB::pgsBCAE(Bacillus subtilis)-tet^{R}, | | |
| | ΔalgD.8.44.KJGXLIVFA | | |
| AZBB711 | Avin_16040::pgsBCAE(Bacillus subtilis)- | | |
| | strep^{R}, ΔalgD.8.44.KJGXLIVFA | | |
| AZBB725 | Avin_16040::pgsBCAE(Bacillus subtilis)- | | 233 mg/L |
| | strep^{R}, ΔalgD.8.44.KJGXLIVFA, ΔphbBAC | | |
| AZBB726 | pgsBCAE(Bacillus subtilis)-strep^{R}(behind | | 128 mg/L |
| | Avin_51340 promoter), | | |
| | ΔalgD.8.44.KJGXLIVFA, ΔphbBAC | | |
| AZBB715 | Disruption of Avin_22170 in AZBB725 | Dark Blue | 343 mg/L |
| Mut#4 | background | | |

### Example 7

In this Example, A. vinelandii strains that had been genetically deregulated for nitrogen fixation by knocking out expression of nifL and nifA were grown using multiple carbon sources, and production of ammonium was measured for each growth condition.

AZBB568 (ΔnifLA, Δnif)

When grown on sucrose, AZBB568 fixed approximately 25 mM ammonium after 96 hours, which is comparable to AZBB163 (A. vinelandii which is partially deregulated for nitrogen fixation) grown in standard B medium (data not shown). Optical density at 600 nm (OD₆₀₀) can be measured to estimate cell confluency, or how robustly the cells are growing. When grown on sucrose, AZBB568 reached an OD₆₀₀ of approximately 3.5.

Pyruvate, a TCA cycle precursor, was almost as effective as sucrose at supporting ammonium production, with levels approaching 24 mM. Growth on pyruvate peaked after 24 hours with an OD₆₀₀ of approximately 1.5.

Malate and 2-oxoglutarate were able to support fairly robust ammonium production as well. Ammonium levels reached about 16 mM and about 10 mM, respectively, after 96 hours. However, cell growth in media containing malate or 2-oxoglutarate was lower than that in media containing sucrose, and the OD₆₀₀ for cells grown in each media remained around 1.0.

Succinate, however, was unable to support ammonium production as levels were only about 1 mM. Nonetheless, AZBB568 grew well on succinate, achieving a maximum OD₆₀₀ of about 3.3 at 72 hours. Interestingly, AZBB568 grown in media containing succinate as the primary carbon source turned bright green. This indicated that the cells were producing high amounts of siderophores. Production of siderophores may indicate that growth on succinate rapidly depletes metals in the medium.

The ability of A. vinelandii to use TCA cycle intermediates and other carbon compounds involved in central metabolism varies widely depending on the compound. Further, the nitrogen fixation and ammonium secretion varies depending on the carbon source utilized by different A. vinelandii strains. A. vinelandii DJ grew well using 2-oxoglutarate, malate, pyruvate, or succinate as a carbon source, but does not secrete ammonium. AZBB568 grew using 2-oxoglutarate, malate, pyruvate, or succinate as a carbon source and did secrete ammonium to various levels. No strain of A. vinelandii has been identified that is able to use citrate, oxaloacetate, or fumarate as a carbon source. A summary of carbon sources that support extracellular ammonium secretion from AZBB568 is shown in FIG. 14.

AZBB695 (ΔnifLA/Δrnf1), and AZBB696 (ΔnifLA/Δfix)

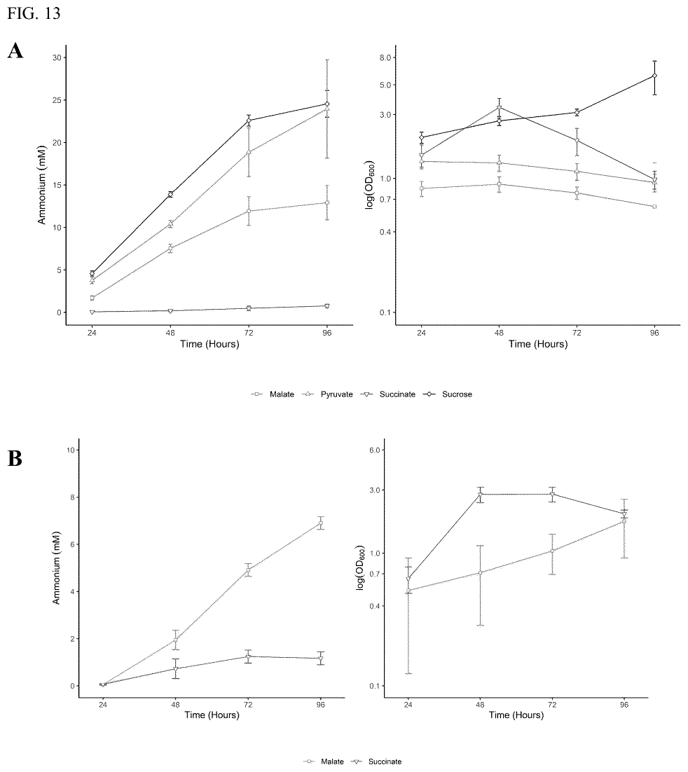

The strains AZBB695 and AZBB696 were derived from AZBB568 to elucidate whether the rnf1 cluster or fix cluster is more important for nitrogen fixation. AZB695, which had the rnf1 cluster removed, but the fix cluster intact, did not produce ammonium on any carbon source tested. Nonetheless, the strain grew well on sucrose, achieving an OD₆₀₀ of >6 after 48 hours (data not shown). Growth on malate was comparable to AZBB568 as well (max OD₆₀₀ ˜1.5). By contrast, AZBB696, which had the fix cluster removed, but the rnf1 cluster intact, still produced as much ammonium as AZBB568 when grown on sucrose or pyruvate. AZBB696 did not produce a significant amount of ammonium on succinate, though it grew to an OD₆₀₀ of approximately 3.4 (FIG. 13A). When grown on maleate, AZBB696 produced levels of ammonium reaching approximately 13 mM. These data suggest a relationship between the rnf1 cluster, overall A. vinelandii fitness, and nitrogen fixation and secretion.

AZBB689 (ΔnifLA/Δrnf1, fixABCX-strep^{R})

Strain AZBB689, which lacked the rnf1 cluster but had the fix cluster expressed behind the S-layer promoter was grown on media containing either maleate or succinate as a carbon source. Both media promoted growth of the strain to an OD₆₀₀ of approximately 2.0 after 96 hours, indicating that A. vinelandii AZBB689 was able to use either carbon source (FIG. 13B). When grown on maleate, AZBB689 secreted almost 7 mM ammonium after 96 hours as shown in FIG. 13B. Growth on succinate resulted in secretion of approximately 1 mM ammonium after 96 hours, indicating that very little nitrogen was fixed under these conditions.

From this Example, it was learned that A. vinelandii strains lacking nifL and nifA grew and fixed nitrogen when provided with 2-oxoglutarate, malate, pyruvate, or sucrose as a carbon source. It was also learned that the carbon source provided influenced the level of nitrogen fixation and secretion.

### Example 8

In this Example, A. vinelandii was grown on different carbon sources commonly found in root exudate. Growth and production of ammonium was measured in each. Both wild-type (DJ) A. vinelandii and the strain AZBB568 were tested with each carbon source.

### Reagents, Strains and Media

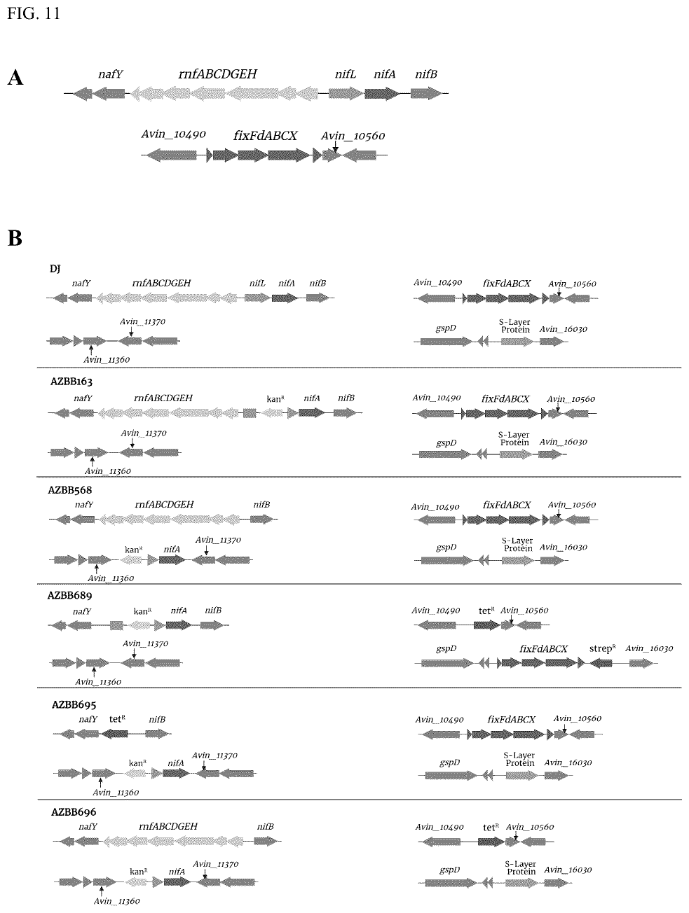

All chemicals were purchased from Sigma-Aldrich (St. Louis, MO) or Fisher Scientific (Pittsburgh, PA), unless otherwise noted. Azotobacter vinelandii DJ (ATCC BAA-1303) was obtained from Dennis Dean (Virginia Tech). Modified A. vinelandii strain AZBB568, described in FIG. 11B, was streaked from a frozen stock onto Burk's medium plates with the appropriate antibiotic and incubated at 30° C. for 3 days to isolate clean colonies. An aliquot of cells (˜20 μL volume) were scraped from the second plate and inoculated into a 125 mL Erlenmeyer flask containing 50 mL of Burk's medium containing elevated Na₂MoO₄, FeSO₄ and Na₂SO₄ (B/MoFeS) medium. B/MoFeS was developed using standard Burk's medium supplemented with 2× iron (18 μM increased to 36 μM as FeSO₄), 5× molybdenum (1 μM increased to 5 μM as Na₂MoO₄), and 3× sulfur (0.8 μM increased to 2.4 μM as Na₂SO₄). When substituted for sucrose, all media contained 2% w/v of carbon source and were adjusted to a pH of 7.2 prior to autoclaving. Cultures were grown at 28° C. with agitation at 180 rpm for four days. Samples were collected approximately every 24 hours and optical density measurements were taken using a Cary 50 Bio Spectrophotometer measuring optical density at 600 nm. For glucose competition experiments, 50 mL of Burk's medium containing glucose substituted for sucrose was prepared and supplemented with varying concentrations of the competing substrate.

### Ammonium Quantification

Cultures were sampled at the same time as optical density measurements were taken, and cells were removed by centrifugation at >12,000×g for one minute. Culture supernatants were either stored at −20° C. or immediately analyzed. Ammonium was quantified using a colorimetric o-phthalaldehyde method. A standard curve was developed using a stock of freshly prepared 0.5 mg/mL (NH₄)₂SO₄. For standard measurements, 900 μL of reagent was combined with a volume of standard ranging from 0 μL to 100 μL. For sample measurements, 900 μL of reagent was combined with up to 100 μL of culture supernatant. All samples had a final volume of 1 mL with deionized water making up the difference in volume as necessary. All measurements were performed with a Cary 50 Bio Spectrophotometer measuring absorbance at 412 nm.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A diazotrophic microbe genetically modified to produce γ-polyglutamic acid.

2. The diazotrophic microbe of claim 1, wherein the diazotrophic microbe comprises one or more exogenous genes.

3. The diazotrophic microbe of claim 2, wherein at least one exogenous gene comprises a gene naturally found in Bacillus spp.

4. The diazotrophic microbe of claim 2, wherein the one or more exogenous genes encode one or more exogenous gene products included in a biosynthetic pathway that produces γ-polyglutamic acid (γ-PGA).

5. The diazotrophic microbe of claim 1, wherein the diazotrophic microbe comprises a gene homologous to a member of the pgsBCAE operon from B. subtilis.

6. The diazotrophic microbe of claim 5, wherein the diazotrophic microbe comprises the pgsBCAE operon from B. subtilis.

7. The diazotrophic microbe of claim 1, wherein the diazotrophic microbe comprises a deletion or disruption of one or more genes involved in the synthesis of a biopolymer.

8. The diazotrophic microbe of claim 7, wherein the biopolymer comprises alginate or polyhydroxybutyrate.

9. The diazotrophic microbe of claim 1, wherein the diazotrophic microbe produces a unique high-molecular weight band on an SDS-PAGE gel as compared to a non-genetically modified diazotrophic microbe.

10. The diazotrophic microbe of claim 1, wherein the diazotrophic microbe is A. vinelandii.

11. A method of increasing growth in an organism, the method consisting of co-culturing the organism with the diazotrophic microbe of claim 1.

12. A diazotrophic microbe genetically modified to exhibit diazotrophic growth when provided with a non-sugar carbon source.

13. The diazotrophic microbe of claim 12, wherein the carbon source is a compound of the tricarboxylic acid (TCA) cycle.

14. The diazotrophic microbe of claim 13, wherein the carbon source is malate, fumarate, succinate, 2-oxaloacetate, or citrate.

15. The diazotrophic microbe of claim 12, wherein the diazotrophic microbe comprises a mutation to a gene homologous to nifLA in A. vinelandii.

16. The diazotrophic microbe of claim 12, wherein the microbe comprises a genetic mutation to increase expression of a gene homologous to the fixABCX cluster from A. vinelandii.


SEQUENCE LISTING (4 sequences)

SEQ ID NO: 1 - DNA, 31 bp, Azotobacter vinelandii
  source 1..31: mol_type = other DNA
taagctagggggaaatgcagacatgttcgga

SEQ ID NO: 2 - DNA, 34 bp, Azotobacter vinelandii
  source 1..34: mol_type = genomic DNA
taagctagttaaggaggtaaaaaaaatgttcgga

SEQ ID NO: 3 - DNA, 36 bp, Azotobacter vinelandii
  source 1..36: mol_type = genomic DNA
ttaatttaatgtaaggtgtgtcaaacgatgaaaaaa

SEQ ID NO: 4 - DNA, 35 bp, Azotobacter vinelandii
  source 1..35: mol_type = genomic DNA
ttaatttaattaaggaggtaaaaaaaatgaaaaaa